(19) United States
(12) Patent Application Publication
(10) Pub. No.: US 2024/0156363 A1
(43) Pub. Date: May 16, 2024

(54) SMART HEART TRACKER

(71) Applicant: Bellabeat Inc., San Francisco, CA (US)

(72) Inventors: Urška Sršen, Zagreb (HR); Sandro Mur, Samobor (HR)

(21) Appl. No.: 18/511,155

(22) Filed: Nov. 16, 2023

Related U.S. Application Data
(60) Provisional application No. 63/425,726, filed on Nov. 16, 2022.

(51) Int. Cl. A61B 5/024; A61B 5/00; A61B 5/0205; A61B 5/11

(52) U.S. Cl. CPC A61B 5/02416; A61B 5/0022; A61B 5/0205; A61B 5/1123; A61B 5/6801; A61B 5/7267; A61B 5/02405; A61B 5/165; A61B 5/4815; A61B 2562/0219

(57) ABSTRACT

A smart heart tracker is described. The smart heart tracker includes a 3-axis accelerometer that measures and stores position and acceleration information for the smart heart tracker and an optical biosensor that measures the heart rate. The smart heart tracker also includes a memory that stores instructions and a processor that executes the instructions to receive the stored position and acceleration information from the 3-axis accelerometer, determine one or more physical activities corresponding to the stored position and acceleration information, and store the determined one or more physical activities.

### TECHNICAL FIELD

The present invention is generally related to the field of health and wellness trackers and methods of using the same. More particularly, aspects of this application are directed to wearables and accompanying products that monitor biometric and lifestyle data to help women better understand how their bodies work and make healthier choices.

### BACKGROUND

Most health and wellness trackers in today's market are focused on monitoring certain specific body indicators, without considering the specifics of the individuals who wear them.

For example, a woman's biological rhythm may be affected by the phases of the menstrual cycle: menstrual, follicular, fertile and luteal. Taking each phase of the cycle into account affects the reproductive system, brain, metabolism, microbiota, immune system, stress response, etc. These menstrual cycle phases are often the biggest impediment when creating healthy wellness routines for women. Hunger, lethargy, mental stress and physical stress are all symptoms that are undesirable. Hence, women's wellness and health needs, as well as exercise strategies, depend on each of the phases.

Accordingly, there is a need for health and wellness trackers that monitor parameters specific to women's needs throughout the menstrual cycle. There is also a need for a specific wellness and exercise program routines that depend on the biometric and lifestyle data collected by the wellness and health trackers that are specific for women's needs. Furthermore, there is a need for a platform that enables use of multiple products and trackers to collect different types of relevant data that are shared across various mobile devices and applications to continuously have up-to-date pictures of women's health and wellness.

### SUMMARY

At least the above-discussed need is addressed and technical solutions are achieved in the art by various embodiments of the present invention. Some embodiments of the present invention pertain to a smart heart tracker shaped and dimensioned to be worn as a piece of jewelry comprising: a 3-axis accelerometer configured to measure and store position and acceleration information for the smart heart tracker; an optical biosensor configured to measure heart rate; a memory configured to store instructions; and a processor communicatively connected to the 3-axis accelerometer and the memory, the processor configured to execute the instructions at least to: receive the stored position and acceleration information from the 3-axis accelerometer; determine one or more physical activities corresponding to the stored position and acceleration information; and store the determined one or more physical activities.

In some embodiments, the smart heart tracker includes a power source configured to provide power to the smart heart tracker.

In some embodiments, the smart heart tracker includes a semiconductor crystal configured to provide a clock signal, wherein the clock signal is used to determine a frequency for measuring and storing the position and acceleration information using the 3-axis accelerometer.

In some embodiments, the smart heart tracker includes a transceiver configured to connect the smart heat tracker to an external device for data communication.

In some embodiments the optical bionsensor obtains a photoplethysmography (PPG) signal.

In some embodiments, the processor is configured to execute the instructions to use the obtained PPG signal in an optical heart rate monitoring (oHRM) algorithm for the measurement of at least one of cardiovascular activity or sleep patterns.

In some embodiments, the processor is configured to execute the instructions use an optical heart rate monitoring (oHRM) algorithm that uses position and acceleration information obtained from the 3-axis accelerometer in combination with data acquired with the obtained PPG signal generate output data related to one or more of activity type, sleep quality, heart rate (HR), heart rate variability (HRV), stress level, or physical state.

In some embodiments, the processor is further configured to execute the instructions at least to: generate a dataset of input features and corresponding output labels from a database of previously stored position and acceleration information; train one or more machine learning classifiers using a first portion of the generated dataset; determine whether the one or more machine learning classifiers are generalized by testing the one or more machine learning classifiers on a second portion of the generated dataset; in a case where the one or more machine learning classifiers are not generalized, validate the one or more machine learning classifiers using a third portion of the generated dataset until the one or more machine learning classifiers are generalized; and in a case where the one or more machine learning classifiers are generalized, determine the one or more physical activities corresponding to the stored position and acceleration information using the generalized one or more machine learning classifiers.

In some embodiments, the input features include signal strength, rhythmicity, and frequency stability, each of which is calculated from the database of previously stored position and acceleration information, and wherein the output labels correspond to the one or more physical activities.

In some embodiments, a smart heart tracker system includes: a smart heart tracker shaped and dimensioned to be worn as a piece of jewelry including: a 3-axis accelerometer configured to measure and store position and acceleration information for the smart heart tracker; an optical biosensor configured to measure heart rate; a transceiver configured to connect the smart heart tracker to an external computing device for data communication, a memory configured to store instructions; and a processor communicatively connected to the 3-axis accelerometer and the memory, the processor configured to execute the instructions at least to: receive the stored position and acceleration information from the 3-axis accelerometer; determine one or more physical activities corresponding to the stored position and acceleration information; and store the determined one or more physical activities; and the external computing device, wherein the external device is configured to be communicatively connected to the smart heart tracker, wherein the external device obtains data from the smart heart tracker, and wherein external device displays information related to the data to a user.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 shows a computing device system, according to some embodiments of the invention.

FIG. 2 shows another computing device system, according to some embodiments of the invention.

FIG. 3 shows a block diagram of a health and wellness platform, according to some embodiments of the present invention;

FIG. 4 shows a block diagram of a smart heart tracker, according to some embodiments of the present invention;

FIG. 5 shows a flowchart of a method of training a machine learning classifier for event detection, according to some embodiments of the present invention;

FIG. 6 shows a diagram of a system for measuring vital signs using illumination, according to some embodiments of the present invention;

FIG. 7 shows a block diagram of a system for heart monitoring, according to some embodiments of the present invention; and

FIG. 8 shows a circuit diagram of an analog front end for an optical biosensor, according to some embodiments of the present invention.

### DETAILED DESCRIPTION

In some embodiments, the systems described herein execute methods for health and wellness tracking and management. It should be noted that the invention is not limited to these or any other examples provided herein, which are referred to for purposes of illustration only.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some embodiments of the present invention may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

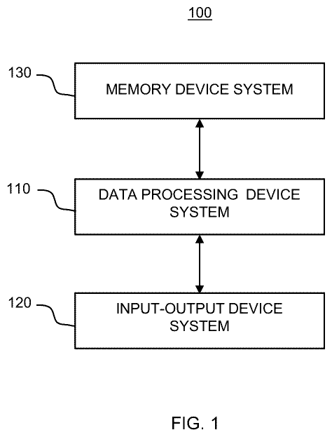

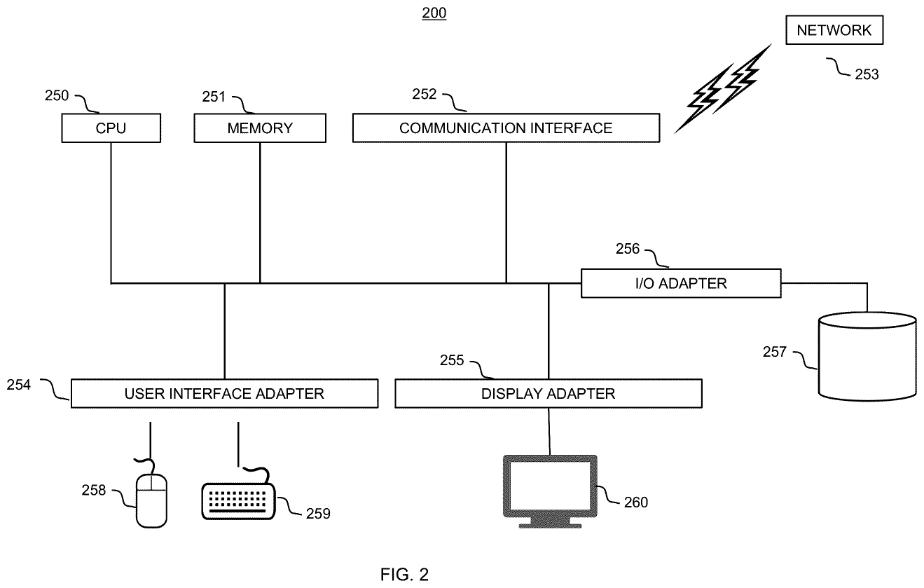

The term “program” in this disclosure should be interpreted to include one or more programs including as a set of instructions or modules that may be executed by one or more components in a system, such as a controller system or data processing device system, in order to cause the system to perform one or more operations. The set of instructions or modules may be stored by any kind of memory device, such as those described subsequently with respect to the memory device system 130, 151, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

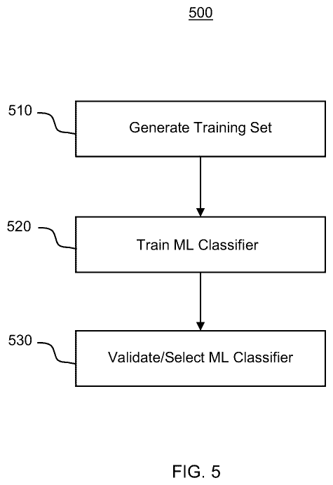

Further still, example methods are described herein with respect to FIG. 5. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangement shown in method FIG. 5 herein is not limited to an actual structure of any program or set of instructions or required ordering of method tasks, and such method 500, according to some embodiments, merely illustrates the tasks that instructions are configured to perform, for example upon execution by a data processing device system in conjunction with interactions with one or more other devices or device systems.

FIG. 1 schematically illustrates a system 100 according to some embodiments. In some embodiments, the system 100 may be a computing device 100 (as shown in FIG. 2). In some embodiments, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various embodiments. Each of the phrases “data processing device”, “data processor”, “processor”, and “computer” is intended to include any data processing device, such as a central processing unit (“CPU”), a desktop computer, a laptop computer, a mainframe computer, a tablet computer, a personal digital assistant, a cellular phone, and any other device configured to process data, manage data, or handle data, whether implemented with electrical, magnetic, optical, biological components, or other.

The memory device system 130 includes one or more processor-accessible memory devices configured to store information, including the information needed to execute the control programs associated with some of the various embodiments. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some embodiments, each of the phrases “processor-accessible memory” and “processor-accessible memory device” is intended to include a non-transitory computer-readable storage medium. In some embodiments, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the embodiments.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the embodiments. In this regard, the input-output device system may include various other devices or systems described in various embodiments.

FIG. 2 shows an example of a computing device system 200, according to some embodiments. The computing device system 200 may include a processor 250, corresponding to the data processing device system 110 of FIG. 1, in some embodiments. The memory 251, input/output (I/O) adapter 256, and non-transitory storage medium 257 may correspond to the memory device system 130 of FIG. 1, according to some embodiments. The user interface adapter 254, mouse 258, keyboard 259, display adapter 255, and display 260 may correspond to the input-output device system 120 of FIG. 1, according to some embodiments. The computing device 200 may also include a communication interface 252 that connects to a network 253 for communicating with other computing devices 200.

Various methods 500 may be performed by way of associated computer-executable instructions according to some example embodiments. In various example embodiments, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various embodiments of methods 500 via interaction with at least, for example, various databases 257 shown in FIG. 2. In these various embodiments, the program may include instructions configured to perform, or cause to be performed, various ones of the instructions associated with execution of various embodiments of methods 500. In some embodiments, methods 500 may include a subset of the associated blocks or additional blocks than those shown in FIG. 5. In some embodiments, methods 500 may include a different sequence indicated between various ones of the associated blocks shown in FIG. 5.

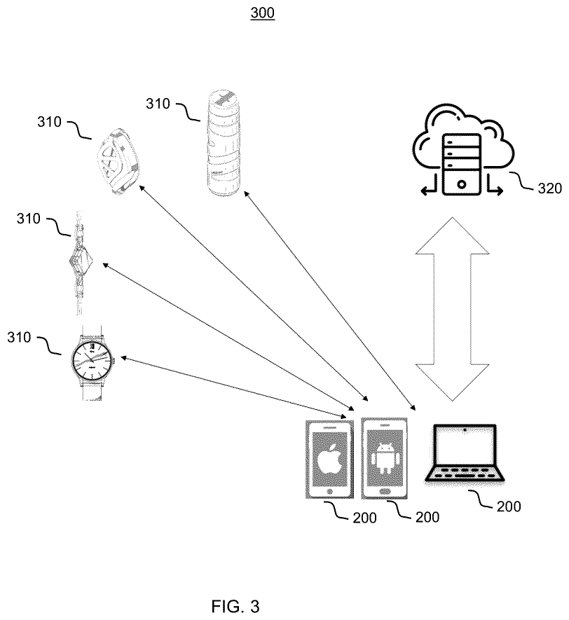

According to some embodiments of the present invention, the system 100 or the computing device 200 includes some or all of the health and wellness system 300 shown in FIG. 3, or vice versa. In this regard, FIG. 3 illustrates a health and wellness system 300, according to some embodiments of the present invention. The health and wellness system 300 may be a particular implementation of the systems 100 or 200, according to some embodiments. The system 300 may include one or more health and wellness trackers 310, which may be in electronic communication with one or more computing devices 200. In some embodiments, the one or more computing devices 200 may be in electronic communications with one or more servers 320. In some embodiments, the electronic communication may be over wired networks, such as the Internet, or wireless networks including wi-fi and Bluetooth.

In some embodiments, the health and wellness trackers 310 include one or more of a smart hybrid watch, a smart necklace, a smart clip, a smart bottle, etc. Other smart health and wellness trackers may also be used to collect health and wellness data from the user. The health and wellness trackers 310 need not have an LCD or LED screen to serve their intended purpose. In some embodiments, the health and wellness trackers 310 are preferably designed to be in the form of ornamental jewelry and to be worn by a user.

The computing devices 200 may include one or more of mobile phones, tablets, laptop or desktop computers, etc. Preferably, the computing devices 200 include at least a display, one or more transmitters and receivers, one or more processors and one or more hard drives on which a computer program with the instructions to receive and process the signals from the health and wellness trackers 310 is stored. When operational, the computer program (application) prompts the user to interact with its one or more health and wellness trackers 310, synchronizes the data stored by the application on the computing devices 200 with the data from the health and wellness trackers 310, receives input from the users, and processes the received data to create additional data points, which are then displayed to the user via a graphical user interface of the health and wellness system 300 (such as the display 260 of the com[putting device 200).

The servers 320 may include one or more cloud servers for back-end processing of information collected from the health and wellness trackers 310 and/or computing devices 200. The servers 320 may also include traditional or in-house data servers. Other servers are also contemplated by this invention for processing data received from the trackers 310 and computing devices 200.

The one or more servers 320 may host programs that synchronize the data on computer devices with the data on the servers that is specific to each user. The back-end platform with servers 320 may share this synchronized data across multiple computer devices 200. For example, if the user has multiple phones (e.g., iOS and Android) used for receiving data from one or more health and wellness trackers 310, the back-end platform may synchronize the data across each computer device 200 so that the user always has current data available across multiple devices.

The back-end platform with one or more servers 320 is further used to process data it may receive from the computing devices 200 and/or health and wellness trackers 310 to customize the user experience. For example, the back-end platform can provide tailored plans for exercise, meals, sleep and meditation schedules etc., which are fully customized based on the data collected from the health and wellness trackers 310, provided by the user, or otherwise provided by the computing devices 200.

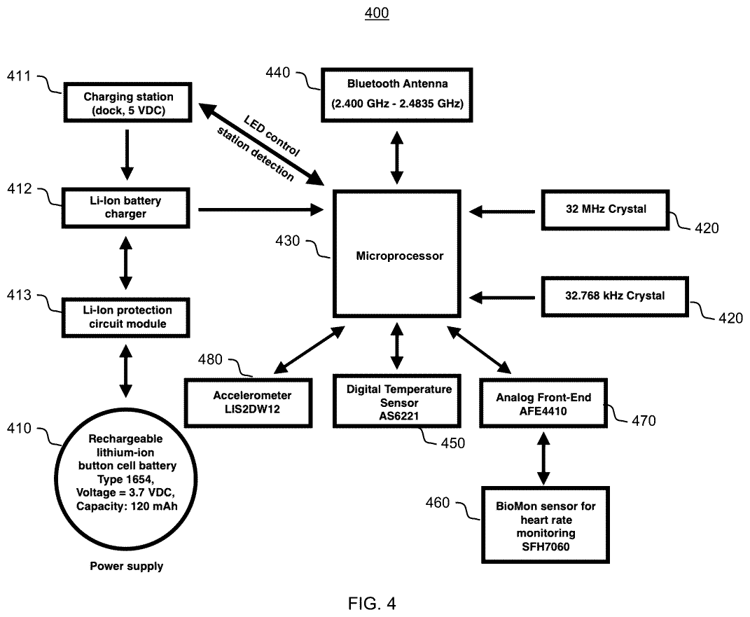

FIG. 4 shows a block diagram of a smart heart tracker 400, according to some embodiments of the invention. The smart heart tracker 400 is an example of a health and wellness tracker 310.

In some embodiments, the smart heart tracker 400 includes a battery 410, one or more semiconductor crystals 420 for providing frequency count, a microprocessor 430, a transceiver 440 (for example, a Bluetooth (BT) chip antenna), a temperature sensor 450, a bio-monitoring sensor 460, an analog front-end 470, and a 3-axis accelerometer 480. In some embodiments, the microprocessor 430 includes a flash memory.

In some embodiments, the smart heart tracker 400 preferably operates as a tracker unit 310 with a 2.4 GHz transceiver 440, but other frequencies may also be used. In some embodiments, the smart heart tracker 400 may connect to a paired computing device 200 via Bluetooth Low Energy (BLE) protocol. Other configurations are also contemplated by this invention to provide a smart heart tracker 400 that operates as a health and wellness tracker 310. In some embodiments, the smart heart tracker 400 may be powered by a rechargeable lithium-ion button cell battery 410, e.g., Type 1654 (Nominal Voltage=3.7 VDC, Nominal Capacity: 120 mAh). The rechargeable battery 410 may be charged with an accompanying charging dock 411 that connects to a port, such as a USB port, on the computing device 200, or via a wall charger, such as a UL-certified USB wall charger (5 VDC). The charging dock includes a battery charger 412 and an overcharging protection circuit module 413, which work together to charge the battery but prevent it from overcharging.

In some embodiments, the smart heart tracker 400 may connect to a paired computing device 200 via Bluetooth Low Energy (BLE) protocol. Other configurations are also contemplated by this invention.

When in use, the smart heart tracker 400 preferably collects certain raw data from the user. For example, the smart heart tracker 400 preferably includes a 3-axis accelerometer 480 that collects data relating to the x, y, and z coordinates of the smart heart tracker 400. This data is collected at a predefined specified rate, for example, 25 Hz (in other words, the data is collected every 40 ms). In some embodiments, the accelerometer 480 can collect any changes in acceleration up to, for example, 8G force. In some embodiments, the accelerometer 480 has an internal storage memory that can collect and store up to a predetermined number of samples (for example, 32 samples). Different sizes of internal storage memory can be used to collect and store fewer or more samples.

In some embodiments, the microprocessor 430 is equipped with a firmware that instructs the microprocessor 430 to request data from the accelerometer 480 at specified time intervals. At a frequency of 25 Hz, that means the microprocessor 430 will request 25 samples every second. In some embodiments, the communication protocol used for the transfer of this raw data from the accelerometer 480 to the microprocessor 430 may include, for example, Serial Peripheral Interface (SPI) protocol, a synchronous serial communication interface specification used for short-distance communication, primarily in embedded systems. Other interface protocols are also contemplated by this invention.

Once the microprocessor 430 has requested the data from the accelerometer 480, the data is transferred via the SPI protocol, and stored in the microprocessor's RAM memory for immediate processing. In some embodiments, the firmware algorithm includes instructions for processing the raw data (for example, the 3D accelerometer signals recorded on the user's wrist), and the microprocessor 430 outputs the calculated output data at a predetermined time interval, for example every second.

In some embodiments, the calculated output data includes one or more of calculated steps (walk or run), calculated activity class (rest, other, walk, run), calculated sleep class (awake, light sleep, deep sleep), etc. Other calculated output data based on the raw data may also be contemplated and implemented in the firmware.

In some embodiments, the calculated output data may be obtained using one or more different classifier algorithms, such as a physical activity classifier, step count algorithm, and step activity classifier, discussed below.

FIG. 5 is a high-level flowchart of a method 500 for training a machine learning-based physical activity classifier using sample data from the smart heart tracker 400. In step 510, the training set of feature vectors and desired output class labels is generated. In step 520, one or more machine learning (ML) classifiers are trained using the generated training set. In step 530, the trained one or more ML classifiers are validated to ensure the ML classifier is generalized to the training set, and the best performing ML classifier, or an ensemble of classifiers, is selected to classify different types of physical activity using data recorded by the smart heart tracker 400.

In some embodiments, a plurality of machine learning algorithms (physical activity classifiers) are trained to classify the user's physical activity. In some embodiments, the accelerometer signals may be used to classify the physical activity using the trained machine learning algorithms, on a sample-by-sample basis. Various features may be used as input features to train the machine learning algorithms. In some embodiment, the input features include signal strength, rhythmicity, and frequency stability, extracted from the accelerometer signals. In some embodiments, these features are used as input features (predictors) to train a machine learning algorithm such as a binary classification tree.

In one embodiment, training of the machine learning algorithm (step 520) is performed using a large database of prior recordings that include a large sample of feature sets (extracted features such as signal strength, rhythmicity, and frequency stability) for a plurality of activities; for example, outdoor walking, outdoor running, road biking, mountain biking, indoor cycling, treadmill walking, treadmill running, and sleeping. In some embodiments, the features are the inputs to the machine learning algorithm and the output nodes of the classifier output the labeled physical activities. In some embodiments, a single machine learning algorithm, such as a random forest of trees, may be used to classify the feature sets into the plurality of activities. In this embodiment, there is an output node for each activity and each output node of the classification tree contains a different likelihood for each activity. Therefore, for each acceleration feature vector sample, the activity classifier returns the most likely activity at any point in time, based on which output node has the highest likelihood. In some embodiments, if the likelihoods of all activities are below a certain value, the output is considered uncertain and the output activity may be ‘other’.

In some embodiments, the training set may be generated (in step 510) by extracting feature vector samples from the raw data while the user is performing a specific activity, and annotating the extracted features with the activity. In some embodiments, the annotations may be performed by one or more experts after the raw data has been collected, until enough labeled samples are collected and a database with extracted features is created.

In some embodiments, a portion of the data is held back from the training set for validation and testing, performed in step 530. The validation dataset is used to give an estimate of a machine learning model's performance while tuning the model's parameters. The test dataset is used to generate an unbiased estimate of the performance of the final tuned model when comparing or selecting between a plurality of machine learning models. It is well known that evaluating the learned models using the training set would result in a biased score as the trained models are, by design, built to learn the biases in the training set. Thus, to evaluate the performance of a trained machine learning model, one needs to use data that has not been used for training.

In one embodiment, the collected data set can be divided equally between the training set and the testing set. The machine learning model is trained using the training set and its performance is evaluated using the testing set. A machine learning model is considered to be generalized or well-trained if its performance on the testing set is within a desired range of the performance on the training set. If the performance on the test set is much worse than the training set, a two-stage validation and testing approach may be used to further generalize the machine learning model.

In some embodiments, in a two stage validation and testing approach, the collected data set is divided between the training set, the validation set, and the testing set. The machine learning model is first trained using the training set, then adjusted to improve its generalization using the validation set, and, finally, evaluated using the testing set.

In some embodiments, the sample data may be divided equally between the desired training, validation, or testing sets. This works well when there is a large collection of data to draw from. In cases where the collection of data samples is limited, other well known techniques, such as leave-one-out validation and testing or k-fold cross validation may be used to perform validation and testing.

In some embodiments, the physical activities may be divided into two categories—rhythmic and non-rhythmic. Examples of rhythmic activities include walking, running, and biking, which are characterized by repeated motions that follow a periodic pattern. For rhythmic activities, a spectral analysis may be performed on the data captured by the accelerometer 480 to identify the frequency of movement. This frequency indicates the cadence at which new steps are occurring; thus, they can be added periodically to a total step count for the recorded activity. However, if the activity is classified as non-rhythmic (for example, rest or other), a time-domain analysis may be performed, in which each oscillation in the data collected by the accelerometer 480 is considered independently. If the oscillation in consideration satisfies a predetermined set of requirements (for example, duration, strength, or shape) the total step count may be increased accordingly; otherwise the oscillation in consideration may be rejected. This method allows for accurate counting of steps (generally associated with rhythmic activity) even when the user is engaged in primarily non-rhythmic activity.

In some embodiments, the firmware of the microprocessor 430 requests data from other sensors, such as the temperature sensor 450 or the bio-monitoring sensor 460, at specified time intervals. The bio-monitoring sensor 480 communicates with the microprocessor 430 via the analog front end 470. In some embodiments, the bio-monitoring sensor 460 is an opto-electronic device that monitors heart rate and measures oxygen saturation using light.

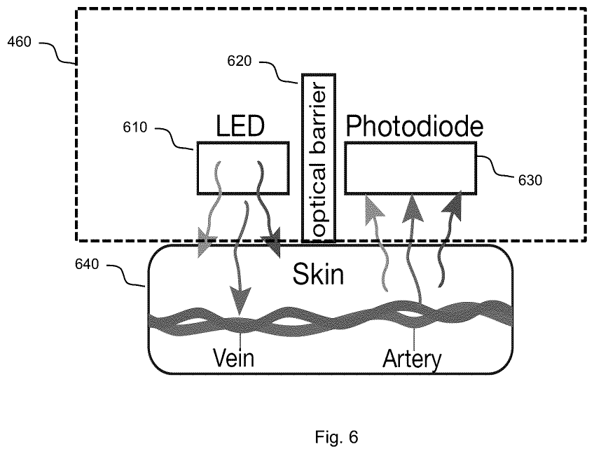

FIG. 6 shows a diagram of an optical front end (bio-monitoring sensor) 460 for measuring vital signs using illumination, according to some embodiments of the present invention. With each cardiac cycle, the heart pumps blood through the circulatory system of the human body 640. Arteries periodically dilate and constrict as blood flows through, and the arteries increase or decrease in volume with every heartbeat. To detect the change in volume caused by the pressure pulse, the skin is illuminated with an LED (light emitter) 610 and the amount of light either transmitted or reflected to a photodetector 630 is measured. The same procedure is used for oxygen saturation measurement. Oxygenated hemoglobin (HbO2) has a significantly different absorption of light than non-oxygenated hemoglobin (Hb). To detect this difference, in some embodiments, the skin is illuminated with, for example, one red and one IR LED light (comprising the LED 610) and the photodetector 630 measures the absorption of light by the blood vessels. The SpO2 value is the quotient of the measured values. In some embodiments, an optical barrier 620 is placed between the LED 610 and the photodetector 630 to prevent leakage of light from the LED 610 to the photodetector 630. The optical barrier 620 ensures that only light reflected from the skin/body 640 is received by the photodetector 630.

In some embodiments, integrated bio-monitoring sensor solutions 460 (emitters 610, barriers 620, and detectors 630 integrated in a single package) may be used instead of discrete components. While both discrete components and integrated solutions are equally efficient and capable of delivering precise and accurate photoplethysmography (PPG) sensing, discrete components (single LEDs and photodetectors) offer more design freedom, because various geometries can be selected and various components can be combined depending on the system requirements. To find the right light source, the LED design and the wavelength of the output light should be considered. Optical barriers 620 between the LED 610 and the photodiode 630 are fundamental to avoid direct cross talk between emitters and detectors. Integrated solutions provide this option and discrete components should be selected accordingly, in order to achieve high-quality measurements.

Integrated bio-monitoring sensor solutions 460, however, also provide several advantages over using discrete components. With integrated solutions, there is no need for an external optical barrier 620, making design and mounting easier and permitting complex customized optical designs. In some embodiments, additional external light barriers 620 may be added to further reduce the optical crosstalk. Further, integrated components generally feature higher light efficacy, with potentially lower power consumption. Moreover, the fixed distance specification between emitter 610 and detector 630 chips leads to good optical consistency in mass production (tolerance controlled on the component level). Finally, a single component integrated device reduces the board space required for mounting and assembly of the emitter/detector pair.

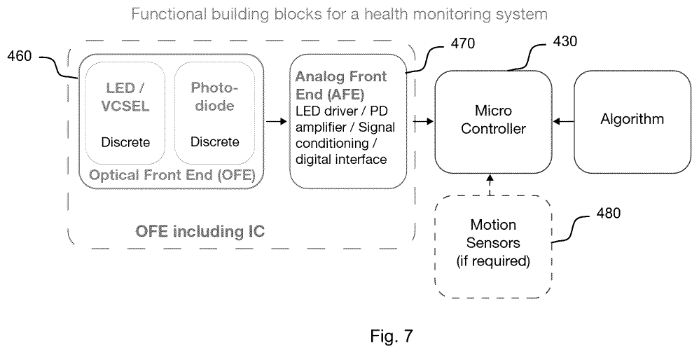

FIG. 7 shows a block diagram of a system for heart monitoring, according to some embodiments of the present invention. In some embodiments, the analog front end (AFE) 470 serves as an interface between the optical front end (OFE) or bio-monitoring sensor 460 and microcontroller 430. The microcontroller 430 instructs the AFE 460 on how to control the OFE 470 in order to acquire the desired raw data for classification (the classification algorithms have been discussed above with respect to data acquired using the accelerometer 480; similar classification algorithms are trained using the biosensor data to predict heart health measures such as heart rate, oxygen saturation etc.). In some embodiments, the microcontroller 430 acquires data from both the OFE 470 and the accelerometer 480 at the same time, and processes it using the firmware algorithm running on the microcontroller 430.

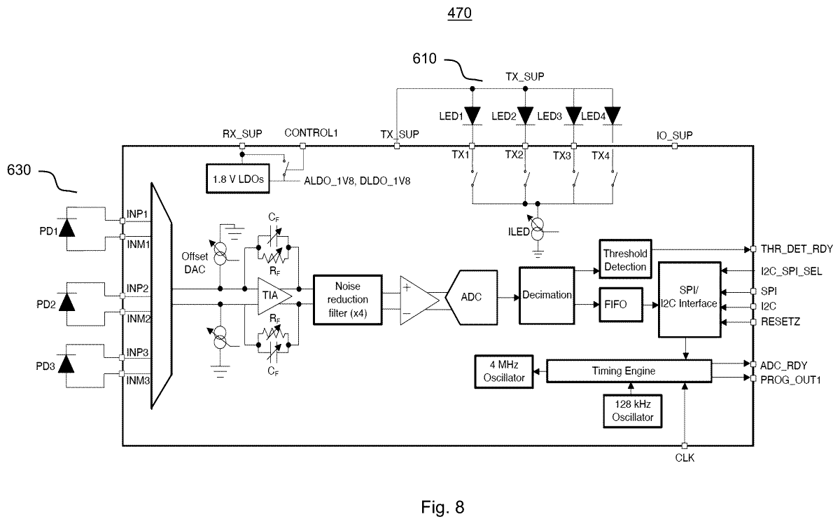

FIG. 8 shows a circuit diagram of an analog front end 470 for an optical biosensor 460, according to some embodiments of the present invention. An example of the AFE 470 is an AFE4410 circuit chip. The analog front-end 470 provides control for optical biosensing applications, such as heart-rate monitoring (HRM). In some embodiments, the AFE 470 can control a plurality of light-emitting diodes (LEDs) 610 and a plurality of photodiodes (PDs) 630. In some embodiments, the AFE 470 converts a current from the photodiode 630 into voltage using a transimpedance amplifier (TIA) and digitizes the converted voltage into a digital signal, output to the microprocessor 430 as the raw data, using an analog-to-digital converter (ADC). In some embodiments of the AFE 470, the ADC code is stored in a 128-sample first in, first-out block (FIFO) with programmable depth. The FIFO block can be read out using either an inter-integrated circuit (I2C) interface or a serial peripheral interface (SPI). In some embodiments, the AFE 470 has a fully integrated LED driver with 8-bit current control to drive the LEDs 610. In some embodiments, the AFE 470 has high dynamic range transmit-and-receive circuitry offering a dynamic range of up to 100 dB that enables accurate heart-rate sensing. The AFE 470 achieves extremely low current levels by operating an ultralow power (ULP) mode set by using an ENABLE_ULP register bit.

In some embodiments, the firmware of the microcontroller 430 instructs the microcontroller 430 on how to control the AFE 470 and when to read out the data from the FIFO block. For example, the AFE 470 may be set up to work at the frequency of 25 Hz. That means the microcontroller 430 processes an interrupt triggered by the AFE 470 every 40 ms; in other words, the microcontroller 430 reads out the raw data from the AFE's FIFO block every 40 ms. The communication protocol used for the transfer of this raw data may be, for example, serial peripheral interface (SPI) protocol, a synchronous serial communication interface specification used for short-distance communication, primarily in embedded systems. Other interface protocols are also contemplated by this invention. For example, inter-integrated circuit (I2C), a synchronous, multi-controller/multi-target, packet switched, single-ended, serial communication bus may be used instead of SPI. I2C is widely used for attaching lower-speed peripheral ICs (integrated circuits) to processors and microcontrollers in short-distance, intra-board communication.

Similar to the case with the accelerometer 480 discussed above, once the microprocessor 430 has requested the data from the FIFO and the data is transferred via the SPI protocol, it is stored in the microprocessor's RAM memory for processing. The firmware algorithm further instructs how the raw data (for example a photoplethysmography (PPG) signal recorded on the user's wrist) is to be processed, and the microprocessor outputs the calculated output data at the frequency rate of 25 Hz (every 40 ms), corresponding to the data acquisition rate.

The raw data obtained from photoplethysmography (PPG) signals enables optical heart rate monitoring (oHRM) in health tracking devices 310 for the measurement of cardiovascular activity, sleep patterns, and other health related metrics. Used in combination with raw data acquired from the movement sensors (such as the accelerometer 480), oHRM may increase the accuracy of the physical activity classifiers discussed above. Stress assessment and management applications use oHRM to measure heart rate variability (HRV), relaxation, and sleep quality. In some embodiments, the monitoring and measurement of these parameters relies on a highly accurate estimation of inter-beat intervals (IBI), i.e., the time between consecutive R-waves as seen on an electrocardiogram (ECG). This, in turn, requires high quality PPG signals coupled with smart detection algorithms.

In some embodiments, the firmware of the microcontroller 430 includes an oHRM algorithm that takes the data from the accelerometer 480 and the biosensor 460 as inputs, and generates output data for activity type, sleep quality, heart rate (HR), heart rate variability (HRV), stress level, and physical state (for example, worn state). In some embodiments, the oHRM algorithm includes a set of states and instructions that enable a pragmatic, low-power estimation of activity, HR, and stress measures. The smart heart tracker 400 seamlessly switches between different modes, thereby reducing sampling frequency when needed and, consequently, prolonging battery life. For instance, the oHRM algorithm may detect whether the smart heart tracker 400 is being worn or not, during the night, based on several signal features extracted from the PPG or the accelerometer data. If the smart heart tracker 400 is detected as not being worn, or if the user is moving substantially during the night, the information may be provided to the application programming interface of the oHRM algorithms so that a decision on not accounting for IBIs during those moments may be made.

In some embodiments, the oHRM algorithm library may evaluate HR during rest and walking/running activity, monitor sleep, and measure Mb for heart rate variability (HRV) and stress analysis. These features allow an accurate estimation of HRV metrics. In addition to these classical measures, a new metric has been developed to capture the coupling between sympathetic and parasympathetic nervous systems: the cardiac coherence. This metric is highly suitable for stress and relaxation measurement, especially during mindfulness exercises.

In some embodiments, the calculated HR output data includes:
HR—Current value of the heart rate in beats-per-minute
CL—Confidence level of the current heart rate estimate (0%400%).

In some embodiments, the calculated HRV output data includes:
peak_to_peak—Interbeat interval between 2 peaks (ms)
mean_IBI—Mean interbeat interval (ms)
mean_resp_period—Mean respiratory period (breaths-per-minute)
coherence—Coherence
sdnn—standard deviation value of the IBI/RR (time between two successive heart beats) over the estimation interval
rmssd—root mean square standard deviation value of the IBI/RR over the estimation interval
sdsd—standard deviation of differences between successive normal cycles of the IBI/RR over the estimation interval
pnn50—Percent of successive normal cycles (NN) that differs by more than 50 ms
pnn20—Percent of successive NN that differs by more than 20 ms
If_power—Power in the LF (low frequency) band
hf_power—Power in the HF (high frequency) band
hrv_reliability—General reliability for the HRV estimation

In some embodiments, the calculated output from the oHRM algorithm that detects whether the smart heart tracker is being worn or not includes:
the device is worn.
the worn detection is in progress
the device is worn and only HR measurement is possible
the device is worn and HR/HRV measurement is possible
the device is not worn.

Other calculated output data based on the raw data is also contemplated in the invention, and may be implemented in the firmware.

In some embodiments, several heart rate variability (HRV) indexes are implemented in the oHRM algorithm. The outputs from the oHRM algorithm include one or more of the following heart rate variability indexes: peak to peak interbeat interval (IBI), mean IBI, mean respiratory period, and cardiac coherence at rest.

In some embodiments, the autonomic nervous system (ANS) represents the neuronal mechanisms involved in the regulation of the body functions. The center of this regulation is the hypothalamus and its control is mediated through sensing (afferent) and acting (efferent) nerves. There are two objectives of controlling the ANS: the adaptation of the body to the current conditions (homeostasis) and the preparation of the body to face hazardous situations (fight or flight). Homeostasis includes the regulation of vital functions (heart rate, blood pressure, temperature, breathing, digestion, etc.) to ensure that the body is in optimal conditions. The fight or flight control is related to the perception of the upcoming challenges that the body will have to face. These regulation mechanisms have been developed in the human body in order to provide the ability to anticipate physical hazards (e.g. the encounter of a predator), and to rapidly prepare the body for physical response; thus increasing the ability to survive. In modern societies, the physical hazardous situations have significantly reduced but the fight or flight mechanism is still present. In some situations, this regulation mechanism is beneficial (e.g. acute stress situation) but a prolonged activity of the mechanism by situations that are perceive has harmful (e.g. stress, mobbing, insufficient rest, etc.) can lead to a degradation of the health status (e.g. burn-out, cardiac problems, digestive problems, etc.).

The function of the ANS is divided into two different control loops: the sympathetic and the vagal controls. The sympathetic control has the function of stimulating the “active” part of body control (increase of heart rate, increase of blood pressure, vase-dilatation of the blood vessels in the muscles, etc.). The vagal control has the function of activating the resting and recovery part of body control (lower heart rate, decrease blood pressure, etc.). The two regulation loops are activated simultaneously and are balanced according to the information of the afferent nerves.

In some embodiments, the state of the autonomic system can be deduced indirectly by the monitoring of the relation between the variations of heart rate (HRV) and the respiration. During the breathing cycles the pressure in the chest is modulated (relatively to the atmospheric pressure) in order to inflate and deflate the lung. These changes of pressure also modulate the return of blood to the heart, and the volume of blood that is ejected by every heart contraction. During the inspiration portion of the breathing cycle, the venous return, and thus the ejected volume, is increased and the blood pressure follows the same trend. This increase of pressure is sensed by the baro-reflex (measure of the dilatation of the aorta) and the information is transmitted to the ANS control through the afferent nerves. The response of the ANS is a decrease of the vagal control that results in an increase of the heart rate and thus a decrease of the blood pressure. During the expiration portion of the breathing cycle, the inverse process is active. Therefore, the duration of the beat-to-beat intervals are modulated by the respiration, and the observed sequence exhibits a sinusoidal oscillation. This interaction between respiration and heart rate is also modulated by the global tone of the ANS. When the user is relaxed, not-stressed, and the breathing rate is low, the vagal control is dominant and the relative amplitude of the modulation of the beat-to-beat intervals will be large. On the other hand, when the user is under stress, tired, or physically active, the sympathetic control is dominant and the respiration related modulation is significantly attenuated or even not present. Therefore, observation of the respiration related variation of the beat-to-beat interval permits an indirect observation of the tone of the control of the autonomic nervous system.

In some embodiments, cardiac coherence measures the interaction between breathing and cardiac variability. As the range of breathing frequency is within the range of frequencies spanned by the vagal control (HF bands in HRV), the analysis of the strength of the coupling between heart rate and breathing offers the opportunity to indirectly measure the vagal status (vagal activity is high when subject is at rest, in non stressed conditions). The measurement of cardiac coherence is a subset of the analysis of HRV. It offers the advantage that valuable information can be extracted from signal duration as short as a few breathing cycles while classical analysis of HRV requires around 5 minutes (with impact on battery).

In some embodiments, two use cases of estimating the cardiac coherence are included in the oHRM algorithm: passive and active. In passive measurement, the measurement of the coherence features is obtained without notice of the user. In active measurement, the user is asked (or requests) to perform a specific task that allows them to estimate an indication of their autonomic status or to improve it.

One suitable moment to passively monitor cardiac coherence is during the night because of two factors: the repeatability and the quasi-absence of movements that ensures the acquisition of good signals of good quality. Generally, the following results are expected when cardiac coherence is estimated passively. When stress level is low and recovery is good, a low cardiac frequency (high mean_ibi), a high modulation rate (high peak_to_peak_ibi and high coherence) of the beat-to-beat intervals and a slow breathing (slow mean_resp_period) rate are expected. Under stress and poor recovery periods, the opposite variation can be expected. The long term monitoring of these values allows the health and wellness system to provide early feedback to the user of a degradation, or to provide the user with motivation when health status improves. The same kind of measurement can be performed during the day when the acceleration measurements, from the accelerometer 480, detect a segment with an absence of movement. However, the repeatability of the measure and, therefore, its interpretation is much more problematic because of the context of the measure (e.g. at rest or after a physical activity).

Active estimation of the cardiac coherence may be performed “on demand”. In these embodiments, the user requests a measurement, assuming that she is at rest. The measurement of the relative amplitude modulation provides direct information about the user's stress level. The user can either breathe normally or deeply, but she has to keep the same breathing style to ensure a repeatability of the measurements. Similarly, “on demand measurements” can be performed before and after relaxing or meditating exercises, to assess their efficiency by comparing the cardiac coherence results before and after the exercises. Finally, the health and wellness system can be used to provide real-time bio-feedback to the user during breathing exercises. The objective of the biofeedback would be, through an ad hoc visual and/or acoustical system, to maximize cardiac coherence and, thus, to improve vagal regulation of the autonomic nervous system.

In some embodiments of the invention, the physical activity classifiers classify the user's movements, based on the recorded data, into different types of activities including, but not limited to, “idle”, “movement_no_walk”, “movement_walk”, “movement_run”, “movement_speed”, “movement_other”, “low activity”, “sleep_light”, and “sleep_deep”. In some embodiments, the classified data, captured by the smart heart tracker 400, is stored in an internal flash memory of the smart heart tracker 400 as an event-per-minute (EPM marker). In some embodiments, the first portion (addressed area) of this allocated part of the flash memory contains a timestamp, which is used by the health and wellness application to reconstruct the exact date and time of the classified activity (events). In some embodiments, each of the EPM markers is 4 bytes long, where the most significant byte is used to identify the activity or event type. The remaining 3 bytes are used as data containers for each specific marker, activity, or event. It is also important to note that the most significant bit (31) determines whether an event belongs to the current minute or the following one.

In addition to physical activity labels, the flash memory may store other EPM markers corresponding to the usage and state of the smart heart tracker 400. For example, a “reset” event indicates the smart heart tracker 400 was restarted. A reset event could be generated by powering on the smart heart tracker 400, changing the battery, performing a hardware reset, or performing a software reset. Other EPM markers include, but are not limited to, “alarm”, “battery”, “Bluetooth_connect”, “bluetooth_disconnect” etc.

The stored post-processed data (for example, EPM markers) may be later collected by the health and wellness system 300 for further processing. For example, when the user opens a client application/graphical user interface of the health and wellness system 300 on a computing device 200, such as a mobile device, or selects to synchronize the data between the trackers 310 and the health and wellness system 300, this stored post-processed data is transferred via Bluetooth protocol to the user's mobile device. An exemplar Bluetooth protocol is Bluetooth Low Energy protocol (BLE 4.2 or later). When this process is completed, the flash memory of the smart heart tracker 400 is erased, and the RAM is cleared.

As explained above, health and wellness system 300 may have a multitude of different health and wellness trackers 310, which collect one or more raw data from the sensors included in the devices worn and/or used by the user. For example, the tracking device 310 may collect raw data from its exemplary PPG sensors, accelerometer and/or scale. The tracking devices may include a firmware that processes this raw data into usable health and wellness tracking data. The translated usable health and wellness tracking data may include e.g.,: Running steps; Walking steps; Other steps; Idle time; Distance; Cadence; Deep sleep; Light sleep; Heart rate; Cardiac coherence; Respiratory rate; Heart rate variability; Battery voltage and remaining life,

The usable health and wellness tracking data (EPM data) is then stored in local memory on the device. The frequency of data collection is at 25 Hz, i.e., the data is collected by the device every 0.04 seconds. The tracking device 310 will continue to collect and store the data until it is synchronized with one of its paired computer devices.

This synchronization may be manual or automatic. The user may manually synchronize the tracker by e.g., double-tapping the tracking device. Alternatively, the synchronization may be automatic, when the tracker connects to the paired computer device (when the software application is used).

As previously pointed out, upon receiving the raw, processed and post-processed data from the health and wellness trackers 310 (tracker data), and/or the user data entered via the graphical user interface on a computing device 200 running a front-end application as described above, the application may instruct the CPU to run various algorithms, which utilize the user data and/or tracking data to compute additional data, which is referred to as “algorithm data.”. The algorithm data may include: Readiness score; Wellness score; Activity time; Heart rate based algorithm; Steps based algorithm; Active calories; Heart rate based algorithm; Steps based algorithm; Period prediction; Sleep segments and duration; Heart rate based algorithm; Steps based algorithm; Stress sensitivity.

This algorithm data, as well as one or more of user data and/or tracking data, may be displayed via a graphical user interface by the application running on a computing device 200.

In this particular example, the user interface shows the wellness score as a number, and further shows some additional data via a daily progress. In particular, the application, based on certain data about the user (age, weight, height, user-specific data, etc.) sets certain recommended goals for the number of steps (running, walking, etc.), other activity (e.g., gym exercise, dancing, cycling, pilates, etc.), mindfulness (e.g., meditation, yoga, breathing exercises, walking in the nature, quiet time, etc.), hydration intake (water consumption) and sleep (naps, or sleep), and then displays the daily progress within the user interface. A more detailed view of the daily overview can be e.g., displayed on a separate screen,

This algorithm, user and tracking data are stored over time for a predefined period, and can be further processed to show different wellness score trends, e.g., over a week.

Aside from a wellness tab showing the wellness score and daily overview and trends relating to other wellness data, the user interface can further provide an interface showing a readiness score and related data For example, the user interface can display the readiness score as a number based on the user's resting heart rate, respiratory rate and cardiac coherence, which may be collected during deep sleep. After e.g., three days of data collection, the first readiness score may be obtained. Preferably, after seven days, the readiness score becomes more accurate, and the more data is collected the readiness score becomes more accurate over time. Most preferably, the full calibration of the readiness score occurs with 30 days of continuous use or longer.

The parameters that go into calculating the readiness score include a heart rate, resting heart rate, respiratory rate and cardiac coherence.

Similarly, the user interface can also allow the user to enter user-specific data, e.g., relating to the menstrual cycle. The application then monitors over time various phases and tends to relating to e.g., cycle length, average period length, etc.

Finally, the application running on a computing device 200 can also provide a user interface where the user can be provided with user-specific content, tailored based on the tracking, user, and algorithm data. This tailored content may be pooled using e.g., artificial intelligence, from a pool of available content in a database. For example, the user interface may provide inspirational videos, exercise routines, recipes, or nutritional plans, specific for the user and the user's current stage (in a menstrual cycle, fitness level, etc.).

The user can also have the ability to selectively browse through the database of various content and select preferred content (e.g., mindfulness exercises, breathing techniques, etc. The application running on a computer device, can then use the user's preferred selection to further tailor the recommended content specific for the user.

It should be obvious to one of ordinary skill in the art that subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A smart heart tracker shaped and dimensioned to be worn as a piece of jewelry comprising:
a 3-axis accelerometer configured to measure and store position and acceleration information for the smart heart tracker;
an optical biosensor configured to measure heart rate;
a memory configured to store instructions; and
a processor communicatively connected to the 3-axis accelerometer and the memory, the processor configured to execute the instructions at least to:
receive the stored position and acceleration information from the 3-axis accelerometer;
determine one or more physical activities corresponding to the stored position and acceleration information; and
store the determined one or more physical activities.

2. The smart heart tracker according to claim 1, further comprising a power source configured to provide power to the smart heart tracker.

3. The smart heart tracker according to claim 1, further comprising a semiconductor crystal configured to provide a clock signal,
wherein the clock signal is used to determine a frequency for measuring and storing the position and acceleration information using the 3-axis accelerometer.

4. The smart heart tracker according to claim 1, further comprising a transceiver configured to connect the smart heat tracker to an external device for data communication.

5. The smart heart tracker according to claim 1, wherein the optical bionsensor obtains a photoplethysmography (PPG) signal.

6. The smart heart tracker according to claim 5, wherein the processor is configured to execute the instructions to use the obtained PPG signal in an optical heart rate monitoring (oHRM) algorithm for the measurement of at least one of cardiovascular activity or sleep patterns.

7. The smart heart tracker according to claim 5, wherein the processor is configured to execute the instructions use an optical heart rate monitoring (oHRM) algorithm that uses position and acceleration information obtained from the 3-axis accelerometer in combination with data acquired with the obtained PPG signal generate output data related to one or more of activity type, sleep quality, heart rate (HR), heart rate variability (HRV), stress level, or physical state.

8. The smart heart tracker according to claim 1, wherein the processor is further configured to execute the instructions at least to:
generate a dataset of input features and corresponding output labels from a database of previously stored position and acceleration information;
train one or more machine learning classifiers using a first portion of the generated dataset;
determine whether the one or more machine learning classifiers are generalized by testing the one or more machine learning classifiers on a second portion of the generated dataset;
in a case where the one or more machine learning classifiers are not generalized, validate the one or more machine learning classifiers using a third portion of the generated dataset until the one or more machine learning classifiers are generalized; and
in a case where the one or more machine learning classifiers are generalized, determine the one or more physical activities corresponding to the stored position and acceleration information using the generalized one or more machine learning classifiers.

9. The smart heart tracker according to claim 8,
wherein the input features include signal strength, rhythmicity, and frequency stability, each of which is calculated from the database of previously stored position and acceleration information, and
wherein the output labels correspond to the one or more physical activities.

10. A smart heart tracker system comprising:
a smart heart tracker shaped and dimensioned to be worn as a piece of jewelry including:
a 3-axis accelerometer configured to measure and store position and acceleration information for the smart heart tracker;
an optical biosensor configured to measure heart rate;
a transceiver configured to connect the smart heart tracker to an external computing device for data communication.
a memory configured to store instructions; and
a processor communicatively connected to the 3-axis accelerometer and the memory, the processor configured to execute the instructions at least to:
receive the stored position and acceleration information from the 3-axis accelerometer;
determine one or more physical activities corresponding to the stored position and acceleration information; and
store the determined one or more physical activities; and
the external computing device,
wherein the external device is configured to be communicatively connected to the smart heart tracker,
wherein the external device obtains data from the smart heart tracker, and
wherein external device displays information related to the data to a user.
